(19) United States
(12) Patent Application Publication
(10) Pub. No.: US 2021/0085857 A1
(43) Pub. Date: Mar. 25, 2021

(54) SYSTEMS AND METHODS FOR PRECISION MATCHED IMMUNOGLOBULIN INFUSION

(71) Applicant: INNOVATIVE HEALTH SCIENCES, LLC, Chester, NY (US)

(72) Inventor: Andrew SEALFON, Chester, NY (US)

(21) Appl. No.: 17/026,050

(22) Filed: Sep. 18, 2020

Related U.S. Application Data
(60) Provisional application No. 62/902,591, filed on Sep. 19, 2019.

(51) Int. Cl. A61M 5/168; A61M 5/162; A61M 5/14

(52) U.S. Cl. CPC A61M 5/16804; A61M 5/162; A61M 2005/1401; A61M 2205/3334; A61M 5/1413

(57) ABSTRACT

Systems, administration sets, and methods of manufacturing for delivering an infusion fluid into a patient's anatomic space include a controller pre-set to deliver a desired flow rate of infusion fluid and an administration set matched to the controller, the administration set includes a pre-determined number of flow tubes having diameters and lengths selected based upon the desired flow rate and number of infusion sites for a specific infusion fluid treatment.

### CROSS REFERENCE TO RELATED APPLICATION

This application claims priority from and the benefit of U.S. Provisional Application No. 62/902,591 filed on Sep. 19, 2019, which is hereby incorporated by reference for all purposes as if fully set forth herein.

### TECHNICAL FIELD

The invention relates generally to systems and methods for precision matched selectable flow rate controllers and needle sets. More specifically, the invention relates to a selectable flow rate controllers and needle sets to deliver fluids for infusion therapy safely and accurately using a constant pressure syringe driver.

### BACKGROUND

Current infusion systems on the market are mostly electrically powered and function by delivering fluids at a pre-set flow rate. In order to maintain the preset flow rate, the system must increase pressure in response to any blockage or other increase in fluid resistance from anywhere in the infusion circuit. This increased pressure can cause severe site reactions, pain, and tissue necrosis. Other infusion systems consist of mechanical syringe drivers, but these generally require a separate flow rate tubing selection for each desired flow rate, which cannot be easily changed once the infusion begins. Still others have a variable flow rate controller for subcutaneous administrations, but it is not calibrated, leaving the flow rate delivered a mystery and complicating the optimization of the infusion treatment.

Other infusion systems include balloon elastomeric pumps that fill an expandable balloon that pushes a drug out through a fixed restrictive tubing set. However, elastomeric pumps have drawbacks, including trapping drug and air in the balloon, having low and variable pressure delivery that is insufficient for several medications, and delivering highly inaccurate flow rates that vary due to temperature. They can also have problems associated with batch-to-batch variability and filling and can be costly to provide to patients. Lastly, past infusion systems include intravenous gravity drip sets, which connect to a large bag of medicine and deliver the medicine/fluid to a vein at very low pressures, but with great inaccuracy in flow rates. These systems require frequent nursing supervision (circa every 15 minutes) to ensure the medicine is properly infusing.

Infusion systems and methods of use administer fluids (generally medications in liquid form) including immunoglobulins for Primary Immune Deficiency Diseases (PIDD) or neuromodulation (neurology), monoclonal antibody therapies for various diseases, hydration, antibiotics, analgesia, and other therapies for other diseases. An infusion pump is a medical device that delivers fluids, including nutrients and medications, including immunoglobulin or antibiotics, into a patient in controlled amounts. The nutrients and medications can include insulin, other hormones, antibiotics, chemotherapy drugs, pain relievers, and other fluids.

Infusion pumps can be used to deliver fluids intravenously, as well as subcutaneously (beneath the skin), arterially, and epidurally (within the surface of the central nervous system). Infusion pumps can reliably administer fluids in ways that would be impractically expensive, unsafe, or unreliable if performed manually by a nursing staff. Infusion pumps offer advantages over manual administration of fluids, including the ability to deliver fluids in very small volumes and the ability to deliver fluids at precisely programmed rates or automated intervals. For example, infusion pumps can administer 1 ml per hour injections (too small a dose for drip methods), injections every minute, injections with repeated boluses requested by the patient (e.g., for patient-controlled analgesia up to a maximum allowed number of boluses over a time period), or fluids whose volumes and delivery vary by the time of day.

Mechanical constant pressure infusion pump systems often use disposable infusion sets to link the pump system to an infusion site of a patient. These sets usually have fixed flow rate tubing between the infusion site and the infusion pump. For constant flow electric pump systems, the tubing is referred to as an “extension set” and has undefined flow properties as the electric pump will adjust to the pressure required to maintain the desired flow rate.

As used herein, “needle set” and “intravenous infusion set” are “administration sets” and refers to the delivery assembly of tubing, luer locks, line locks, flow rate controllers, needles, and needle safety features (e.g., butterfly or disc). The “tubing set” refers to the tubing used in the “needle set” and “intravenous infusion set.”

Further, in conventional mechanical infusion systems, separate flow rate restriction tubing is used to create different flow rates for different drugs, intravenous catheters, or subcutaneous needle sets based on the requirements of the infusion rate for the patient. There are currently 22 offerings in the market for precision flow rate tubing sets. Each precision flow rate tubing set includes a set length and a specific diameter provided by the manufacturer. In the case of subcutaneous applications, assuming the same drug is used, each precision flow rate tubing set produces a different flow rate that is dependent upon the number of needle sites used in the needle set, and the diameter and length of tubing and needle used. Subcutaneous needle sets are provided in configurations of 1-8 needles grouped together into a common manifold with each configuration requiring a different series flow rate tubing that may differ in either length and/or diameter. Additionally, in these known systems, there are generally four bore sizes of needles (28 g, 27 g, 26 g, 24 g) which also result in different flow rates with each precision flow rate tubing set. These flow rates are calculated using a flow rate calculator or a mobile app to enter system parameters (e.g., specific fluid viscosity, etc.) to calculate infusion flow rate and time. For intravenous administrations, most of the drugs are low viscosity, and the intravenous catheters do not impair the flow rate accuracy at lower flow rates (<120 ml/hr). Also, mechanical infusion pumps currently on the market target subcutaneous administrations, ignoring the fact that about 80-90% of all infusions are intravenous.

One example of a variable flow rate controller is described in U.S. Patent Application Publication 2016/0256625. The variable flow rate controller replaces the need for multiple fixed flow rate tubing sets, which minimizes stocking issues. However, it was found that the variable flow rate controller was unpredictable with great flow rate inconsistencies and loss of accuracy at both the low-end and high-end settings. Additionally, these controllers had an unrestricted flow rate at the wide-open maximum setting (i.e., the markings do not directly indicate the flow rate). Thus, when using these systems, clinicians have a difficult time predicting or knowing what the actual delivered flow rates are likely to be. As each infusion is unique, it becomes a clinical challenge to know whether any problems during administration exist in the patient or in the variable flow rate controller device. Without an established baseline, it is difficult to diagnose and correct any infusion complications.

In mechanical constant pressure systems, components in direct or indirect contact with the fluid path influence the final flow rate delivered to the patient. Any part of the system can contribute to an incorrect flow rate being delivered to the patient and the associated harmful adverse reactions that can occur to the patient.

While some adverse treatment events may be the result of user error, many of the reported adverse events with previous systems are related to deficiencies in infusion system design and engineering, with the risk usually being an excessive flow rate or high output pressure. The additional calculations required for each variation of needle and tubing sets and controllers adds unneeded complexity and points of error. These deficiencies create problems themselves or contribute to user error by manifesting themselves in improper flow rates of the infusion fluids at the patient infusion sites.

The above information disclosed in this Background section is only for understanding of the background of the inventive concepts, and, therefore, it may contain information that does not constitute prior art.

### SUMMARY

The infusion systems constructed, and the methods performed, according to the principles and exemplary implementations of the invention address one of more the above-noted deficiencies. For example, infusion systems constructed according to the principles and some exemplary implementations of the invention (and methods implementing the same) deliver infusion fluid to a patient using a matched variable flow rate controller and an administration set and a constant pressure syringe driver for delivery of the infusion fluid. In one example implementation of the invention, a precision matched infusion system delivers immunoglobulin for subcutaneous applications. In another example implementation of the invention, a precision matched infusion system uses a constant pressure syringe driver and a matched variable flow rate controller and tubing set to deliver an antibiotic infusion for intravenous applications.

In one exemplary implementation of the invention, a calibrated disposable infusion set is used to ensure that the controller delivers the correct flow rate. By constructing and using a calibrated flow rate controller and compatible parts of the flow circuit, systems in accordance with the invention safely and accurately deliver infusion fluids to the patient.

Infusion systems and methods in accordance with the principles and some exemplary implementations of the invention solve many of the major issues of pharmaceutical drug delivery problems. They can drastically improve safety by limiting pressure to safe values. They are much less labor intensive, as they obviate the need for numerous fixed rate tubing sets. Infusion systems and methods in accordance with the principles and some exemplary implementations of the invention can provide these benefits at a much lower price point and may be scalable for manufacture and thus can meet the demands of new infectious viruses like COVID 19. They can be used by clinicians or trained patients, in a hospital, clinic, or at home. Infusion systems and methods in accordance with the principles and some exemplary implementations of systems and methods in accordance with the invention also can provide direct indication of the flow rate—what you see is what you get—and require no calculations, Excel spread sheets, or long lists of tables for referencing the flow rate output for each situation. They can eliminate the need for a range of different flow rate controls, can be automatically calibrated to provide the correct flow rate indications for any number of needle sites, and eliminate errors while improving the sterile compliance by connecting all infusion components together in one package. Infusion systems and methods in accordance with the principles and some exemplary for subcutaneous delivery of immunoglobulins and intravenous delivery of antibiotics can deliver the maximum flow rates of drugs currently on the market and can meet future demands for even faster flow rates. There are currently no systems available on the market that can provide the flexibility, safety, ease of use, and overall infusion performance at a low-cost price point as with infusion systems and methods in accordance with the principles and some exemplary implementations of with the invention.

For example, matching a variable flow rate controller with either an intravenous or subcutaneous administration set solves many of the problems in the art. Intravenous tubing sets are matched and packaged with a variable flow rate controller as a calibrated infusion set. Similarly, in subcutaneous infusions, a subcutaneous needle set is matched and packaged with a variable flow rate controller as another calibrated infusion set. The matched sets are delivered in sterile packages, and several major advantages over prior systems are realized.

These advantages include fewer items to stock, repeatable and accurate flow control settings, and vastly improved patient and caregiver safety. Infusion systems and methods in accordance with the principles and some exemplary implementations of the invention can provide pre-set maximum flow rates (set at the factory or by the health care provider), the number of needle sets may be matched based on a maximum flow rate setting. This improves patient safety as it obviates prior methods of connecting the controller to a needle set (for subcutaneous applications) and eliminates a source of potential contamination in all applications by reducing the chance of sterility contamination.

To circumvent the inconsistencies and inaccuracies of current market offerings, some exemplary implementations of the invention are specifically calibrated to ensure that the controller delivers the precise flow rate, which is clearly indicated on the controller dial for patients and clinicians. Additionally, since the controller enables patients and/or providers to select various flow rates, the need for additional fixed rate tubing sets (current market offerings) is unnecessary. This enables a tailored infusion experience for each patient according to their treatment regimen.

For subcutaneous applications, the more needle sites used, the greater need for higher flow rates from the variable flow rate controller. For example, if the maximum flow rate value used with a four-needle set was used with a single needle set, the delivered rate to the patient would be excessive and would cause discomfort. Conversely, if the maximum flow rate for a single-needle set is used with a four-needle set, the flow rate per site will be well below the maximum flow rate permitted, and the patient will not be able to receive the treatment in the most time efficient manner. Further, matching flow rate controllers constructed in accordance with some exemplary implementations of the invention can correctly account for flow rates at the extreme settings of the controllers and label the flow rate produced, in ml/hr, with a visual reference, so patients are fully aware of the safe range of flow rates.

Exemplary implementations of the invention can provide specific cost advantages over known systems, such as the variable flow rate controller in the U.S. Patent Application Publication 2016/0256624, by simplifying stocking of the needed variable flow rate controller. This avoids the need to stock multiple different variable flow rate controllers. In addition, there is less labor for the health care provider, as they can provide a single matched package with all components that the patient needs. Additionally, reducing the decision-making process and complications when changing needle sets or tubing sets or variable flow rate controllers greatly reduces user errors.

Additional features of the inventive concepts will be set forth in the description which follows, and in part will be apparent from the description, or may be learned by practice of the inventive concepts.

According to one aspect of the invention, an infusion system for delivering an infusion fluid into a patient's anatomic space includes: a controller pre-set to deliver a desired flow rate of infusion fluid; and an administration set matched to the controller, the administration set including a pre-determined number of flow tubes having diameters and lengths selected based upon the desired flow rate and number of infusion sites for a specific infusion fluid treatment.

The administration set may include a needle set to subcutaneously deliver the infusion fluid into the patient's anatomic space, and the needle set further may include a pre-determined number of needles having diameters selected based upon the desired flow rate, a number of infusion sites to subcutaneously deliver the infusion fluid into the patient's anatomic space, and the specific infusion fluid to be delivered.

The infusion system further may include a substantially constant pressure infusion driver to deliver the infusion fluid; and the pre-determined number of needles may be pre-calibrated to deliver a predetermined flow rate of the specific infusion fluid at a predetermined infusion fluid pressure based on the number of needles in the administration set, a flow rate of the flow tubes, and the specific infusion fluid to be delivered.

The number of needles in the administration set may include one to eight.

The controller may be configured to be attached to the flow tubes and pre-set to deliver a pre-set flow rate less than or equal to a maximum flow rate for the specific infusion fluid treatment.

The flow tubes and the needles may be packaged in a single-use package.

The administration set may include an intravenous infusion set to intravenously deliver the infusion fluid into the patient's anatomic space, and the intravenous infusion set further may include a tube to receive the infusion fluid from the infusion driver; and a connector to receive the infusion fluid from the controller and the tube to deliver the infusion fluid to an IV bag or catheter at a predetermined flow rate; and the predetermined flow rate may be selected for the specific infusion fluid at a predetermined infusion fluid pressure, and a flow rate of the tube.

The controller may be configured to be attached to the system and pre-set to deliver a pre-set flow rate less than or equal to a maximum flow rate for the specific infusion fluid treatment.

The connector may include a luer lock connector.

According to another aspect of the invention, an infusion system for delivering an infusion fluid into a patient's anatomic space includes: a pump driver to deliver the infusion fluid into the patient's anatomic space at a substantially constant pressure and a desired flow rate; an administration set to deliver the infusion fluid into a patient's anatomic space, and the administration set includes: a pre-determined number of flow tubes having diameters and lengths selected based upon the desired flow rate, and number of infusion sites for a specific infusion fluid treatment.

The administration set may include a needle set to subcutaneously deliver the infusion fluid into the patient's anatomic space, and the needle set further may include: a pre-determined number of needles having diameters selected based upon the desired flow rate, a number of infusion sites to subcutaneously deliver the infusion fluid into the patient's anatomic space, and the specific infusion fluid.

The pre-determined number of needles may be pre-calibrated to deliver a predetermined flow rate of the specific infusion fluid at a predetermined infusion fluid pressure based on the number of needles in the administration set, a flow rate of the flow tubes, and the specific infusion fluid to be delivered.

The number of needles in the administration set may include one to eight.

The driver may be configured to be attached to the flow tubes and pre-set to deliver a pre-set flow rate less than or equal to a maximum flow rate for the specific infusion fluid treatment.

The flow tubes and the needles may be packaged in a single-use package.

The administration set may include an infusion set to intravenously deliver the infusion fluid into the patient's anatomic space, the administration set further may include: a connector to receive the infusion fluid and to deliver the infusion fluid to an IV bag or catheter at a predetermined flow rate selected for the specific infusion fluid treatment at a predetermined infusion fluid pressure based on a flow rate of the flow tubes; and a flow rate controller to be attached to the connector and pre-set to deliver a pre-set flow rate less than or equal to a maximum flow rate for the specific infusion fluid treatment.

The connector may include a luer lock connector.

According to another aspect of the invention, a method of manufacturing an infusion system for delivering a specific infusion fluid to a patient's anatomical space includes the steps of: matching a flow rate controller to an administration set, where the flow controller is pre-set to deliver a desired flow rate of infusion fluid and the administration set includes a predetermined number of flow tubes having lengths and diameters based on the desired flow rate and number of infusion sites for the specific infusion fluid treatment.

The administration set may include a needle set to subcutaneously deliver the infusion fluid into the patient's anatomic space, and the method further may include: selecting a pre-determined number of needles having diameters selected based on the desired flow rate, a number of infusion sites to subcutaneously deliver the infusion fluid into the patient's anatomic space, and the specific infusion fluid.

The method of manufacturing further may include: configuring and pre-calibrating a number of needles to deliver the infusion fluid into the patient's anatomic space, and determining a flow rate of the specific infusion fluid at a pre-determined infusion fluid pressure based on the number of needles in the administration set, a flow rate of the flow tubes, and the specific infusion fluid to be delivered.

The method further may include: configuring the flow rate controller to be attached to the flow tubes; and pre-setting the flow rate controller to deliver a pre-set flow rate less than or equal to a maximum flow rate for the specific infusion fluid treatment.

The method further may include packaging the flow tubes and the needles in a single-use package.

The number of needles of the infusion system may include one to eight.

The infusion system may be configured to intravenously deliver the infusion fluid into the patient's anatomic space, and the method further may include configuring a tube to receive the infusion fluid from an infusion driver; and configuring a connector to receive the infusion fluid from the matched flow rate controller and the tube to deliver the infusion fluid to an IV bag or catheter at a predetermined flow rate selected for the specific infusion fluid treatment at a predetermined infusion fluid pressure based a flow rate of the tube.

The method further may include configuring the flow rate controller to be attached to the connector; and pre-setting the flow rate controller to deliver a pre-set flow rate less than or equal to a maximum flow rate for the specific infusion fluid treatment.

The method further may include providing an infusion driver to deliver the infusion fluid at a substantially constant pressure.

According to another aspect of the invention, an administration set for delivering an infusion fluid into a patient's anatomic space includes a pre-determined number of flow tubes having diameters and lengths selected based upon a desired flow rate of a controller and a number of infusion sites for a specific infusion fluid treatment.

The administration set further may include a controller pre-set to deliver a desired flow rate of infusion fluid, and the administration set may be matched to the controller.

The administration set further may include a pre-determined number of needles having diameters selected based upon the desired flow rate, a number of infusion sites to subcutaneously deliver the infusion fluid into the patient's anatomic space, and the specific infusion fluid to be delivered.

The administration set further may include a tube to receive infusion fluid from a source of infusion fluid; and a connector to receive infusion fluid from the controller and the tube to deliver the infusion fluid to an IV bag or catheter at a predetermined flow rate selected for the specific infusion fluid at a predetermined infusion fluid pressure and a flow rate of the tube.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate exemplary embodiments of the invention, and together with the description serve to explain the inventive concepts.

FIG. 1A is an illustration of an exemplary embodiment of an infusion system constructed according to the principles of the invention for delivering infusion liquid subcutaneously to a patient.

FIG. 1B is an illustration of another exemplary embodiment of an infusion system constructed according to the principles of the invention for delivering infusion liquid subcutaneously to a patient.

FIG. 2 is a chart of the flow rates, tube sizes, and needle sites used for different drugs to illustrate the need for different variable flow rate controllers for different drugs and needle sites.

FIG. 3 is an illustration of an exemplary embodiment of an infusion system constructed according to the principles of the invention for delivering infusion liquid intravenously to a patient.

FIG. 4A is a perspective view of a variable flow rate controller for use with an infusion system constructed according to the principles of the invention.

FIG. 4B is a cross-sectional view of a variable flow rate controller of FIG. 4A.

FIG. 4C is a cross-sectional view of a variable flow rate controller of FIGS. 4A and 4B showing a decreasing channel and an inlet hole that acts against a slip washer to allow different positions of channels to achieve differing flow rates.

FIG. 5A is a top perspective view of an exemplary embodiment of a butterfly wing constructed according to the principles of the invention shown in an open configuration.

FIG. 5B is a top perspective view of an exemplary embodiment of a butterfly wing with needle constructed according to the principles of the invention.

FIG. 5C is a side sectional perspective view of a butterfly wing of FIG. 5B.

FIG. 5D is a side sectional view of another exemplary embodiment of a butterfly wing with needle using a ball-and-pivot joint constructed according to the principles of the invention.

FIG. 5E is an exploded perspective view of a butterfly wing with needle of FIG. 5B.

FIG. 6A is a perspective view of an exemplary embodiment of a constant pressure syringe pump constructed according to the principles of the invention.

FIG. 6B is a perspective view of a constant pressure syringe pump of FIG. 6A without a cover.

FIG. 6C is a top sectional view of a constant pressure syringe pump of FIG. 6A.

FIG. 6D is an exploded view of a constant pressure syringe pump of FIG. 6A.

FIG. 7 shows a set of calibrated flow dials of a variable flow rate controller of FIG. 4A.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

### Subcutaneous Infusion Example

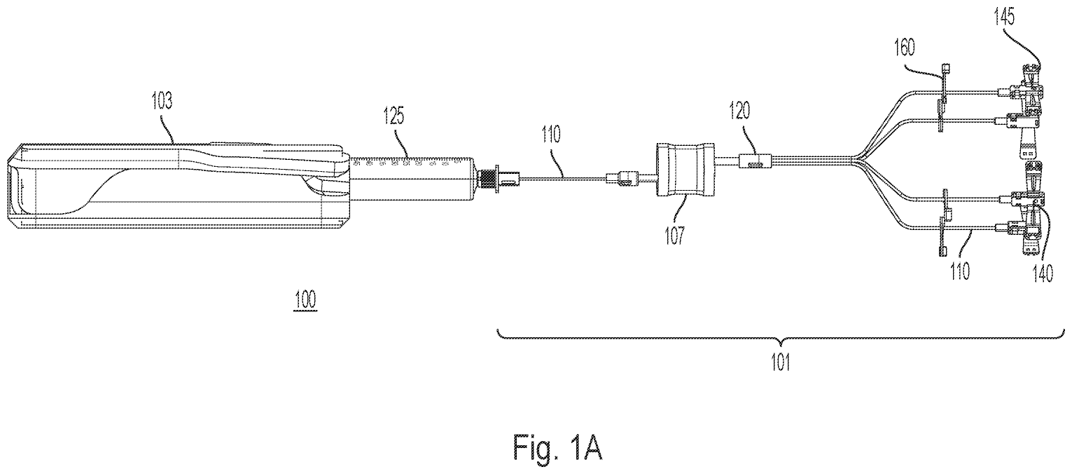

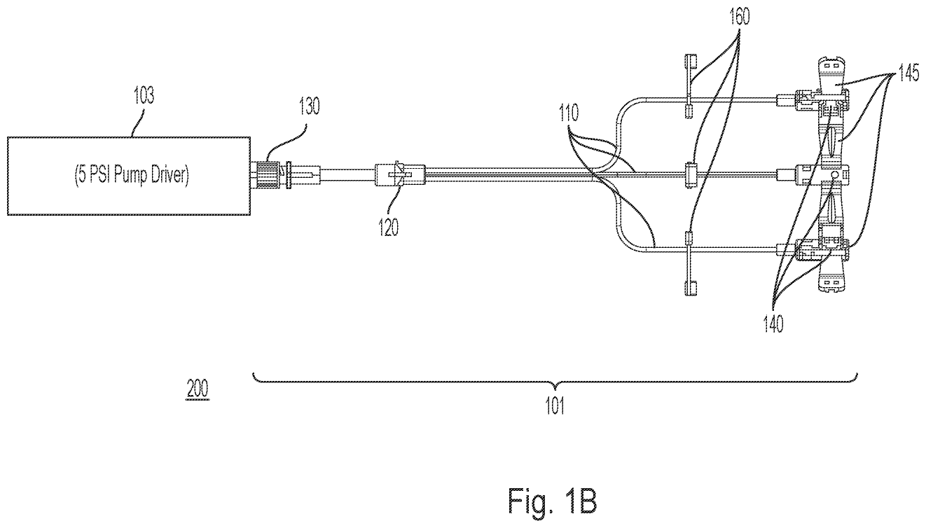

FIG. 1A shows an exemplary embodiment of an infusion system 100 constructed according to the principles of the invention for delivering the infusion liquid subcutaneously to a patient. The infusion system 100 includes an infusion driver 103 with infusion reservoir 125 and infusion needle set 101.

In some exemplary embodiments, as shown in FIG. 1A, the infusion system 100 may be provided to users with the infusion pump 103, which may be a constant pressure syringe driver. The syringe driver 103 is selected based on a need for a particular pressure or amount of liquid over time. The syringe driver (pump) 103 includes a syringe or liquid reservoir 125 and driver which drives the syringe to force the fluid in the reservoir into the infusion needle set 101. The infusion system 100 further includes an infusion subcutaneous needle set 101. The infusion subcutaneous needle set 101 includes a variable flow rate controller 107, needle set series tubing 110, manifold 120, tubing clamp/line lick 160, butterfly connectors/discs 145, and needles 140.

In some exemplary embodiments, the infusion system 100 is provided to users with only an infusion needle set 101 for use with a patient's own separate infusion driver or pump. In some exemplary embodiments, the infusion driver or pump may be connected with the infusion needle set 101 through any known means, including, e.g., a standard luer disc connector.

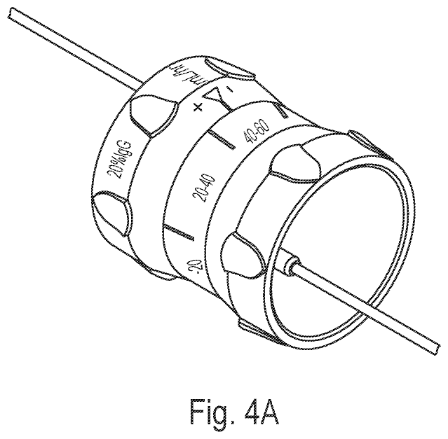

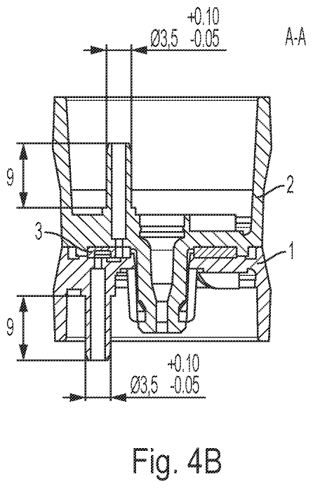

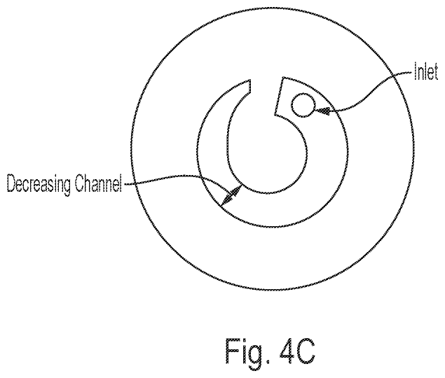

Due to the fluidics of the infusion driver 103, for subcutaneous administrations, as the number of injection sites is increased, the maximum flow rate per site requires an increased flow rate setting for the controller (shown in FIGS. 4A-C). Thus, the number of needles in the needle set combination requires a different series flow rate regulation. As the number of injection sites increases, the series flow rate equivalent must also increase to regulate and maintain the desired flow rate at the injection sites. In one example, a variable flow rate controller and series precision flow rate tube that is set for the maximum flow rate for use with four-needle sites would create an excessive flow rate beyond the manufacturer's approved drug labeling if used for a single needle site.

In the past, to provide particular flow rates in conventional constant pressure infusion systems, a medical professional would have to change the series tubing 110. That is, the medical professional would have to select different series tubing with a larger diameter/length or a smaller diameter/length. This involves selecting another administration set that may not be immediately available and/or may introduce contamination concerns. Exemplary embodiments of the invention, however, provide advantages as users have access to a variable flow rate controller and can select a number of needle sites to provide or adjust the flow rate of the infusion system. The range of flow rates available with the variable flow rate controller and number of needle sites eliminates the need for stocking specific fixed flow rate administration sets and extension sets and eliminates contamination concerns involved in replacing administration sets or connecting extension sets.

To address this issue, in one exemplary embodiment, a system constructed according to the principles of the invention provides a selection of a different flow rate inlet series tubing to control the maximum flow rate of the system based on the number of needle sites required. Thus, providing a user some additional ways to adjust the flow rate. In one example, as the number of needle sites increases, the flow rate required also increases in order to reach the maximum flow rate at each needle site as stated in the drug manufacturer's package insert. A user of the infusion system may adjust the flow rate controller as well as change the infusion needle set to one allowing a higher flow rate based on the increased number of needle sites.

In the exemplary embodiment of the invention shown in FIG. 1A, the flow rate controller 107 with changed series tubing 110 is used to maximize flow rates of the infusion fluid. In FIG. 1A, the variable flow rate controller 107 has flow rates that are marked in segments of low rates (green or 0-20 ml/hr), medium rates (yellow or 20-40 ml/hr) and high rates (red or 40-60 ml/hr) as shown in FIG. 4A. In this exemplary embodiment, the flow rate controller 107 of FIG. 4A is marked for use only with 20% IgG solutions. In other exemplary embodiments, the variable flow rate controller 107 has flow rates marked at increments of 10 ml/hr (e.g., 10, 20, 30, 40, 50, and 60 ml/hr).

In another exemplary embodiment of the invention, the system may change flow rate controllers 107 for Hizentra® and Cuvitru® or other immunoglobulins for subcutaneous applications based on their infusion rates and viscosity. In other exemplary embodiments, the infusion needle set 101 is selected based on the infused drug, treated health issue, syndrome, or disease, desired flow rate, and number of infusion sites. The flow rate controller 107 and needles 140 are directly connected through the needle set tubing 110 to prevent removal and change of parts of the infusion needle set 101. The needle set tubing 110 extends from the variable flow rate controller 107 to a manifold 120 where the needle set tubing 110 divides into individual needle tubes 110 to needles 140. The needle set tubing 110 includes tubing clamps 160 between sections of tubing, e.g., tubing clamps/line locks 160 on each needle tube and/or the tubing between the variable flow rate controller 107 and manifold 120. The needle set tubing 110 does not include luer connectors due to the infusion needle set 101 being a single piece set for users to select based on situation, e.g., number of infusion sites, infusion fluid viscosity, patient comfort, and infusion fluid maximum infusion flow rate. In some exemplary embodiments, the system is used for a neuromodulation treatment that is subcutaneously administered.

In the exemplary embodiment of the invention shown in FIG. 1A, variable flow rate controller 107 is connected to the series tubing 110 with luer connectors. In some exemplary embodiments, the variable flow rate controller 107 and tubing sets 110 are combined into a single package and connected directly to one another with no middle luer connectors. In other words, the needle set 101 has its own dedicated variable flow rate controller 107.

In some exemplary embodiments, the needles 140 include a butterfly or disc assembly 145 for each needle 140 or some variant of butterfly-less and butterfly assembly 145 including needles 140. The infusion needle set 101 generally includes a number of needles 140 between one and eight, however, the number of needles 140 may be greater based on future infusion site allowances and/or changes to needle design. The needles 140 include needles of different bore sizes and lengths, angles of entry, and also are selected for the infusion needle set 101 based on pain control and comfort for a particular patient.

In some exemplary embodiments, the needle(s) 140 of infusion system 100 are inserted into a patient's anatomical space to deliver an infusion fluid. The needle set 101 selected for use is based on a selected infusion fluid and a number of infusion sites. A user or clinician provides a needle set and sets a variable flow rate controller of the needle set to less than or equal to a maximum flow rate of the infusion fluid to be delivered to the patient's anatomical space.

FIG. 1B shows an exemplary embodiment of the infusion system 200 constructed according to the principles of the invention for delivering infusion liquid subcutaneously to a patient. In the exemplary embodiment, the infusion system 200 includes a pump (driver) 103 and an infusion needle set 101. The pump (driver) 103 may be any infusion pump that is able to generate at least about 5 psi of pressure for the infusion fluid flow and includes an infusion fluid reservoir. In one exemplary embodiment, the pump (driver) 103 may be the same infusion driver 103 of FIG. 1A. The infusion needle set 101 includes a luer connection device 130, tri-connector (manifold) 120, needle tubes 110, slide clamps 160 on each tubing set 110, needles 140, and butterfly wing assemblies 145 for each needle 140. The pump (driver) 103 is connected to the needle set 101, similar to the connection between the infusion driver 103 and infusion needle set 101 of FIG. 1A, via the luer connection device 130. The infusion system 200 is similar to infusion system 100, except the infusion needle set 101 lacks a flow rate controller.

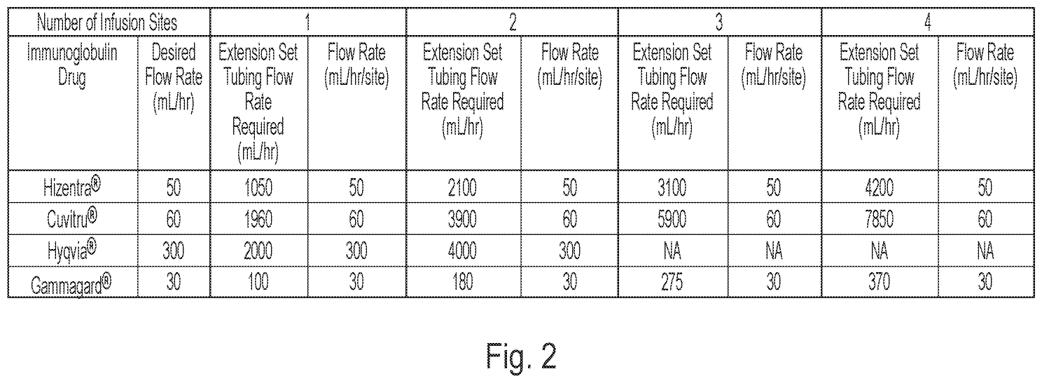

FIG. 2 is a chart of exemplary, calculated subcutaneous flowrates required by each drug, quantity of needle sites, to achieve the flow rates for drugs such as Hizentra®, Cuvitru®, Hyqvia®, or Gammagard® immunoglobulin requiring flow rates between 25 and 300 ml/hr. The infusion system 100 directly provides the same combinations of flow rate selections presented in FIG. 2. For example, specifically for Hizentra® (requiring a flow rate of 50 ml/hr/site), when using a single-needle set, an equivalent flow rate of F1050 is needed. However, if the patient using Hizentra® requires a faster flow rate and/or four-needle sites of infusion, to achieve the same flow rate of 50 ml/hr/site would require an equivalent flow rate of 4200. These custom maximum settings could be either factory set or set by the clinician. The extension set tubing flow rate required flow rate numbers' e.g., 4200, 1050 ml/hr, etc. represent the theoretical water free flow rate required to deliver drug flow rate using a 26G needle as stated in FIG. 2.

Other drugs of different concentrations and/or viscosities will require different flow rate controllers to limit maximum flow rates dependent on the drug's viscosity. For example, in another exemplary embodiment of the invention, the system 300 may include a particular flow rate controller for Vancomycin or other antibiotics for intravenous applications, which would decrease the required stock of fixed flow rate administration sets by health care providers.

In other exemplary embodiments of the invention, different variable flow rate controllers 107 are required for different situations, dependent on the viscosity of the drug used, which results in changes to the labelling of the flow rate controller for different treatment protocols for neuromodulation versus PIDD, to limit the flow rate to the maximums for each treatment protocol.

### Intravenous Infusion Example

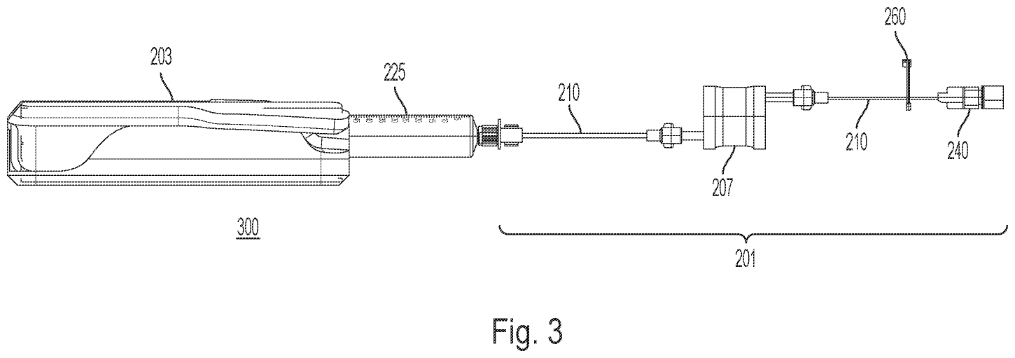

FIG. 3 shows an exemplary embodiment of an infusion system 300 constructed according to the principles of the invention for delivering infusion liquid intravenously to a patient. The infusion system 300 including an infusion intravenous tubing set 201 and infusion driver 203 with infusion reservoir 225. The infusion intravenous set 201 including series tube 210, a variable flow rate controller 207, and a distal luer connector 240 to connect to an IV bag or catheter (not shown separately). The exemplary embodiments of FIG. 3 are similar to those of FIG. 1A above, except as related to the needles and butterfly wings.

### Variable Flow Rate Controller

In FIG. 4A, the variable flow rate controller 107 used with the infusion systems 100 and 300 in some exemplary embodiments of the invention may include custom flow rate controls on the flow rate controllers 107 to set minimum and maximum flow rates or single flow rates. Two inner wheels connected to the main rotational shaft have the ability to set a maximum flow rate and a minimum flow rate. This is accomplished with a series of pin settings (similar to those used to control electric timers), a gear system which disengages from the main drive for setting the flow rate controller, or two settable discs (similar to those used on electric timers on/off controls). In some exemplary embodiments, these controls are lockable using a restricted key design, so that any settings made by the factory or by the clinician cannot be changed by the patient. However, limiting the patient access may be unnecessary because the set range would be, in some exemplary embodiments, safe for patient control.

This flow rate controller is best understood by visualizing the turning shaft of the main controller body is connected to a disc with adjustable slots to impinge upon a fixed shaft on the bottom controller body that can change flow rates in either direction, where one direction further opens/increases the flow rate, and the other direction closes/decreases the flow rate. Further, these slots can be adjusted such that no motion is permitted above or below from a desired flow rate setting, thus turning the variable flow rate controller 107 into a fixed rate controller delivering only a single fixed flow rate.

In particular, as shown in FIGS. 4B and 4C, the variable flow rate controller 107 shows two reciprocal halves of the controller body mounted together on the main shaft with the disc in between such that both end of the slots can be adjusted to any position within the 350-degree rotation limits of the two outside parts of the controller. As a user turns the main controller body, it impinges on the gasket that further impinges on the decreasing channel (shown in FIG. 4C) to limit the flow or increase flow (when rotated in the opposite direction).

Both ends of the slots are adjustable to minimum and maximum values and can be placed so that no interference in the rotation occurs or that the rotation is totally limited to one position or flow rate desired, turning the variable flow rate controller 107 into a single rate fixed system.

In some exemplary embodiments, the variable flow rate controller 107 includes color coded markings for different ranges of flow rates. Thus, more clearly indicating the actual flow rate at the patient through the infusion needle set 101. These indicators may include ranges such as 0-20 ml/hr, 20-40 ml/hr, and 40-60 ml/hr for subcutaneous applications. Further, the indicators may be color coded for green, yellow, and red, respectively to represent low, medium, and high flow rates and potential use danger zones.

In some exemplary embodiments, the variable flow rate controller 107 includes color coded markings for different ranges of flow rates ranging from about 5-about 300 ml/hr for intravenous applications. Further, the indicators may be color coded for green, yellow, and red respectively to represent low, medium, and high flow rates and potential use danger zones.

In some exemplary embodiments, a system 100 includes special packaging that allows infusion providers to adjust the flow rate ranges while maintaining sterility of the infusion needle sets 101. Since the variable flow rate controller 107 is in the same package as the administration needle sets 101 or tubing sets 110, a double pouch arrangement is designed to allow the clinician to adjust the flow rate ranges or single flow rate without jeopardizing the sterility of the needle sets or tubing sets. This unique packaging isolates the needle sets 101 or tubing sets 110 from a separate compartment housing the variable flow rate controller 107, which permits access to the settings.

In some exemplary flow rate controller embodiments, a variable flow rate controller 107 includes different lock-on labelling for specialty flow rate markings. The controllers may include custom flow rate markings for different ranges or for specific drug deliveries. These bands may snap into place either at the factory or by the clinician as desired.

In some exemplary flow rate controller embodiments, a variable flow rate controller 107 includes a keyed locking mechanism, which allows the variable flow rate controller to be delivered either in a fixed flow rate, or in fixed flow range.

In some exemplary embodiments, a variable flow rate controller 107 will be pre-set to the maximum flow rate range of the highest flow rate needed for each combination of needle sets. This results in different settings as more needles are required, since higher flow rates are needed to deliver the liquid to the patient at a set flow rate. This also prevents creating flow rates too fast for a single-needle or a two-needle set.

In some exemplary embodiments, the needle sets use a 26 g needle with 0.036 in+ tubing. In some exemplary embodiments, the connectors have even larger dimensions. In some exemplary embodiments, the tubing includes soft tubing.

In one exemplary embodiment, a variable flow rate controller 107 is set to different ranges but used only for specific treatments and needle sets. For example, for PIDD, one range can be limited to 2400 ml/hr while for a four-needle set for Cuvitru and in another instance, the range of the variable flow rate controller 107 is set at a maximum of 5600 ml/hr with a four-needle set and set to 3200 ml/hr for a two-needle set. In other words, the system would be limited for safety and changeable by the Infusion Provider as needed.

In one exemplary embodiment, a variable flow rate controller includes a channel (FIG. 4C) of variable width and circular length, and by an outside ring rotating around the channel. The flow rate controller can be used to select different channel widths and lengths, which result in different flow rates. By controlling the depth, width, and length of the channel, a wide range of different flow rates can be generated from a single (variable flow rate) controller. The input flow arrives from a series tube on one side of the controller and the output is delivered out the other side of the controller. The variable flow rate controller includes a sliding mating sealing washer and “0” rings to prevent leakage around the channel and rotating shafts.

In some exemplary embodiments for subcutaneous infusion systems, the system packaging includes a complete variable flow rate controller 107 and needle set in one package to provide a single sterilized assembly and luer lock fitting to the pump of the syringe driver. In some exemplary embodiments for intravenous infusion systems, the system packaging includes a complete variable flow rate controller 107 and tubing set in one package, to provide a single sterilized assembly and luer lock fitting to the syringe driver.

FIG. 4B shows an exemplary variable flow rate controller 107 with (1) the interface between the two halves that select the channel location as the one side (2) is rotated into different positions with respect to (3).

As outlined above, FIG. 4C shows a cross-sectional view of the variable flow rate controller 107 with the disc and main controller body not showing. The cross-sectional view shows a channel which decreases in width to limit or increase the fluid flow. The decreasing channel and an inlet hole that acts against a slip washer to allow different positions along the decreasing channel to achieve different flow rates. FIG. 4C shows a decreasing channel (width) in one half the controller, which is selected by rotating the controller halves to select different points in the channel path. The channel varies by width and depth and is then selectable by length to obtain any desired flow rate setting.

Infusion systems and methods in accordance with some exemplary embodiments of the invention accurately and reproducibly deliver an infusion fluid to a patient at a desired anatomical location by allowing for direct control of the infusion system pressure. Patients and clinicians can determine the infusion system flow rate and deliver a volume of an infusion liquid at a speed that does not cause discomfort. Patients and clinicians and other users can match the infusion liquid and needle sites (for subcutaneous applications) and variable flow rate controller settings to increase the probability of safe treatment using the infusion system. A patient or clinician can set these system variables and immediately determine which treatment configuration is best for the treatment type.

### Butterfly Wing Assembly

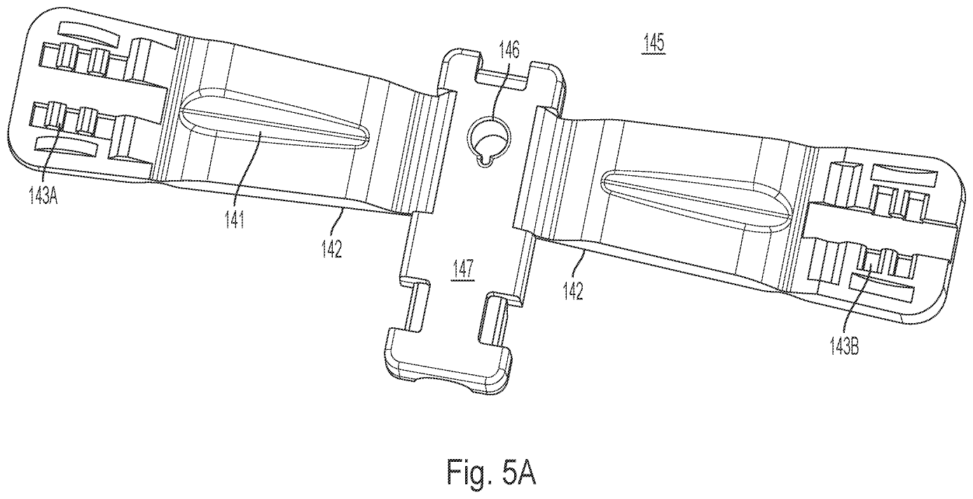

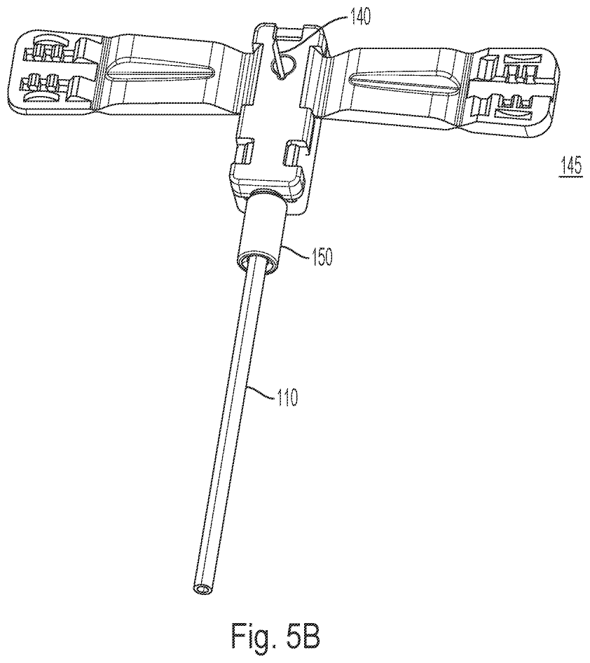

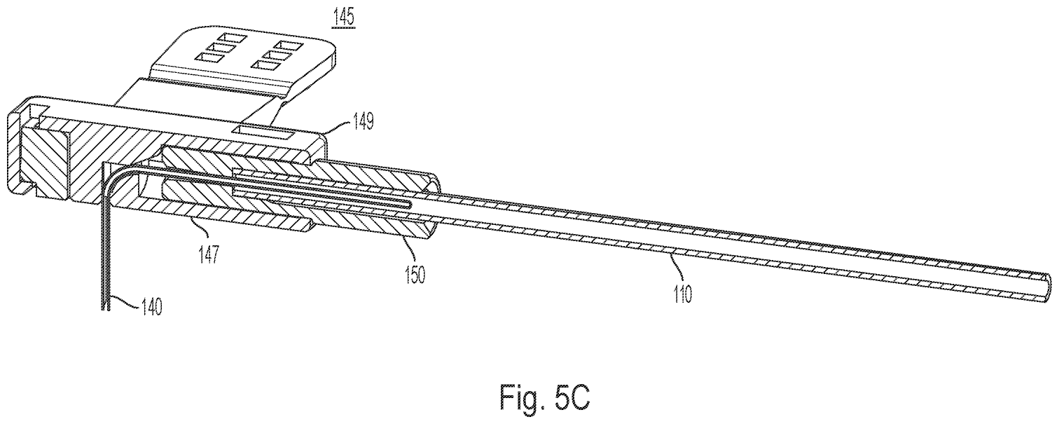

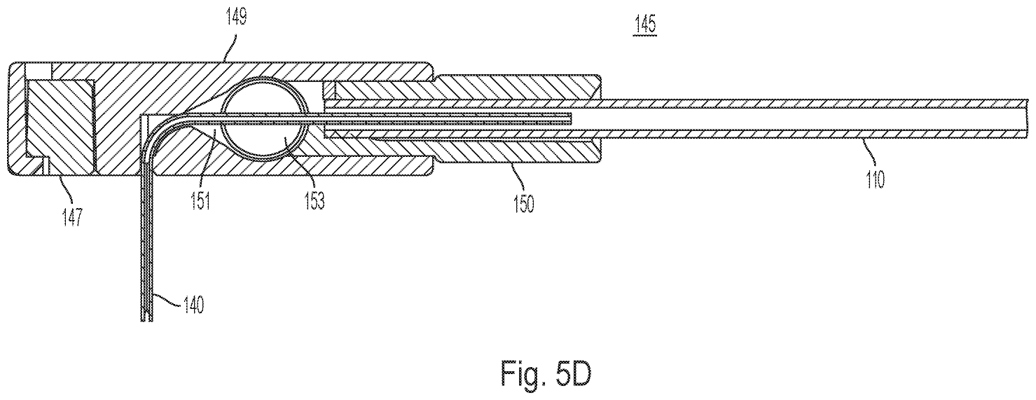

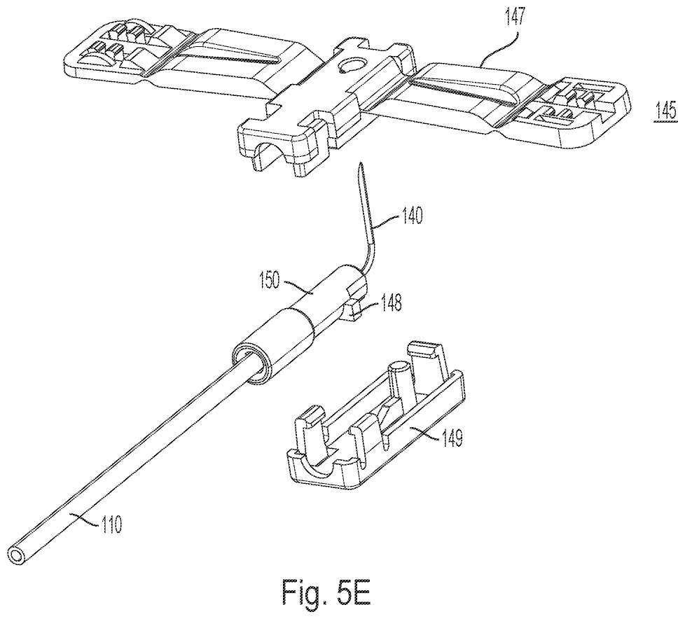

FIG. 5A shows a top perspective view of an exemplary embodiment of a butterfly constructed according to the principles of the invention in an open configuration. The exemplary embodiment of the butterfly 145 includes a needle sleeve protection portion 141, tab 143A and slot 143B connections, and needle access opening 146. The butterfly 145 is connected in series and in the same direction as the length of the series tubing. The butterfly 145 houses a needle 140 such that the needle protrudes both orthogonally to the long axis of the butterfly and to the series needle tubing. In one exemplary implementation, the needle 140 may be bent to achieve this orthogonality. Furthermore, the butterfly housings (FIG. 5A) have symmetrically positioned butterfly wings 142. The butterfly wings 142 are used as a needle insertion/removal handling feature and conform to the patient's skin without causing irritation or discomfort. The butterfly wings 142 also protect the needle after use to eliminate potential harm (e.g., needle-stick injuries). To protect the needle after use, the butterfly wings 142 use needle sleeve protection portion 141. Upon closing the butterfly 145, the needle sleeve protection portion 141 that is about/around the length of the needle 140, encloses the needle tip. This closing mechanism includes a double latch, in which both butterfly wings 142 have a latch configuration to mate with the opposing wing. In this exemplary embodiment, the latch is one or more tabs 143A and one or more slots 143B mechanism(s) which mate together to hold the butterfly 145 in a closed state. When the butterfly wings 142 are closing, users will observe a tactile and/or audible click indicating to users that the butterfly wings 142 are closed, and the needle tip is protected (after use of the needle set). Furthermore, the surface topography of the butterfly wings 142 and its closing mechanism avoid the use of any guiding or latching mechanisms at the periphery of the wing and increases the surface area that contacts the patient during use to reduce discomfort and pain when placed on the skin. In addition, the closing mechanism acts as a guiding feature to guide the butterfly wings 142 together when closing. This prevents misalignment and makes it easier to cover and protect the needle.

The butterfly wings 142 can also include grooves designated to guide and maintain the needles' orthogonal (90°) orientation such that the needle is straight and undamaged when received by the user. This ensures that the needles do not fail to penetrate to the correct skin tissue depth as a result of an angled needle, and the associated discomfort and pain from improper penetration is eliminated.

In other exemplary embodiments, such as illustrated in FIG. 5D, the butterfly 145 in combination with needle 140 can include a ball-and-pivot or floating ball mechanism such that when inside of the butterfly housing 147, the needle 140 may rotate (e.g., five degrees) in any direction at the pivot socket (point) 151. The ball-and-pivot mechanism includes a ball 153 which mates with the needle 140 to hold a portion of the needle 140 in position while allowing rotation, within the pivot socket 151, of the interfaced ball 153 and needle 140. In this fashion, slight motion of the butterfly does not transmit to the needle and does not cause the needle to move within a patient's tissue. As a result, needles in accordance with some exemplary embodiments of the invention eliminate motion forces transmitted through the needle during an infusion, which can otherwise damage tissue and cause pain and inflammation. The pivoting needle feature eliminates tissue damage and pain by rotating the needle at the pivot and within the butterfly housing in response to forces placed on the butterfly.

FIG. 5B shows a top perspective view of an exemplary embodiment of a butterfly wing with needle constructed according to the principles of the invention. As shown, in the exemplary embodiment, the needle 140 is mated with the butterfly 145. The needle 140 includes a needle seat or connector 150 that holds the needle in place while placed in the butterfly 145. The needle 140 connects to the rest of the needle set 101 via the needle (series) tubing 110. FIG. 5C shows a side sectional perspective view of an exemplary embodiment of a butterfly wing with needle constructed according to the principles of the invention. As shown in the exemplary embodiment, the needle seat 150 is placed between a butterfly cover 149 and butterfly housing 147 to hold the needle in place when placed in the butterfly 145. As shown also in FIG. 5E, the cavity between the butterfly cover 149 and butterfly housing 147 further includes a needle holding and guiding path and space to trap the needle seat 150. FIG. 5E shows an exploded perspective view of an exemplary embodiment of a butterfly wing 145 with needle constructed according to the principles of the invention. As shown in the exemplary embodiment, the needle seat 150 may further include spacers 148 that mate with the butterfly housing 147 and/or butterfly cover 149 to hold the needle 140 in a position for the needle sleeve protection portion to protect the needle when the butterfly wing is in a closed state.

### Infusion Driver

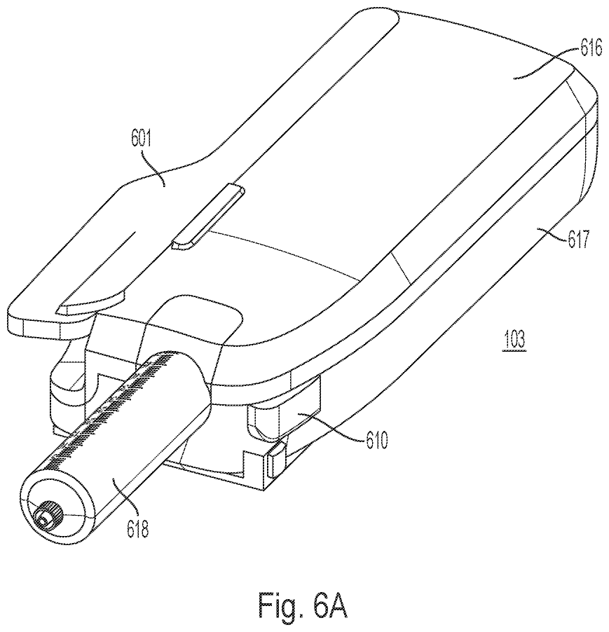

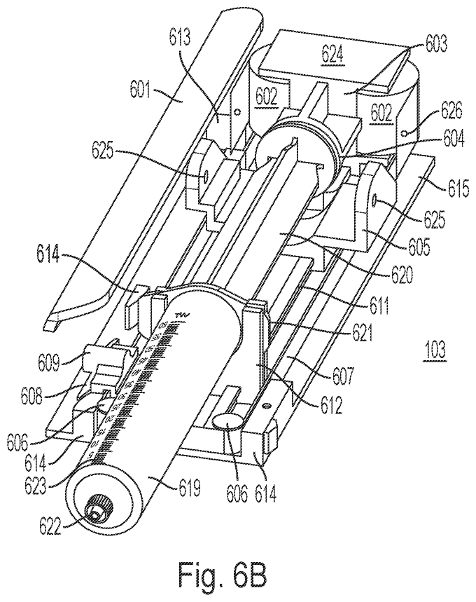

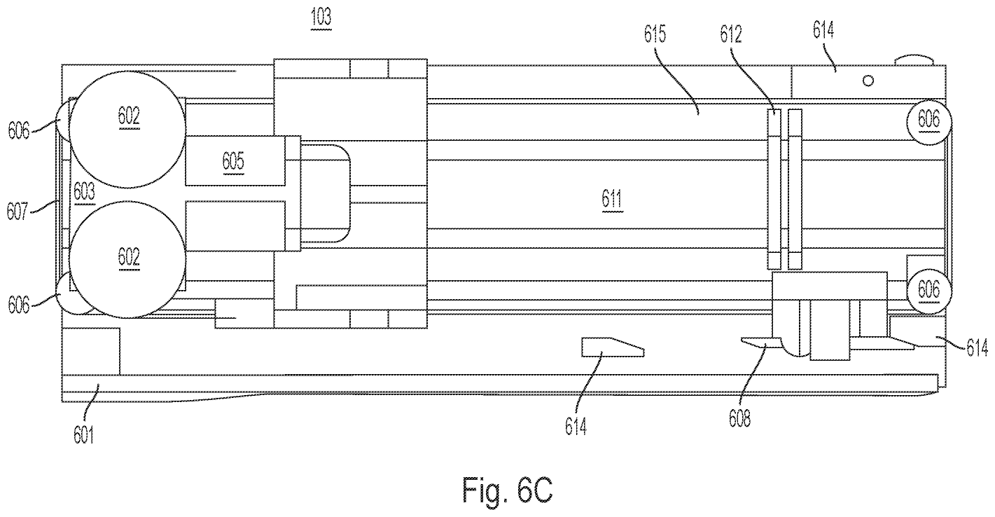

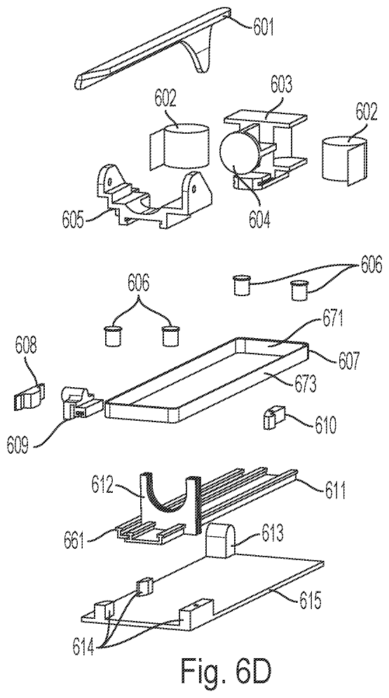

FIG. 6A shows a perspective view of an exemplary embodiment of a constant pressure syringe pump according to the principles of the invention. In one exemplary embodiment of the constant pressure syringe pump 103, the pump 103 includes a syringe 618 acting as a reservoir and including a mechanism for dispensing infusion fluid from the syringe 618 (i.e., the syringe plunger 620 as shown in FIG. 6B). The pump 103 also acts as a housing for the syringe 618. The body of the housing including a main body portion 617 and cover 616. Further, the pump 103 includes an open button 610 to remove the cover 616 from the body 617, and a lever 601 to actuate the pump 103 and dispense infusion fluid from the syringe 618.

FIG. 6B shows a perspective view of an exemplary embodiment of a constant pressure syringe pump according to the principles of the invention without a cover. The constant pressure syringe pump 103 includes a mechanism for mating with the syringe plunger 620 to accurately actuate the syringe plunger 620.

As shown in FIGS. 6A-6D, an exemplary embodiment of the constant pressure syringe pump 103, when not in use or when the lever 601 and cover 616 are closed against the body casing 617 of the pump 103, the pump 103 is in its most compact form. To operate the pump 103 in this condition, a user must first engage a cover opening button 610 that allows the lever 601 and cover 616 to open to some degree.

In an exemplary embodiment of the constant pressure syringe pump 103, the pump 103 actuating mechanism is a lever 601. The lever 601 is attached to the lever attachment point 613 that is fixed to one corner of the base plate 615. The lever attachment point 613 protrudes from the base plate 615 such that the attached lever 601 can rotate around the lever attachment point 613. In some exemplary embodiments, the lever 601 is at a length such that 4 strokes at approximately 3.5 pounds of force per stroke is needed to fully load the pump 103 actuating mechanism. In some exemplary embodiments, the cover 616 may also be attached at the lever attachment point 613 and rotate to some degree. Further, a mechanism (e.g., a spring), can be used to aid in opening the lever 601 and cover 616, such that when the pump 103 is in a “not in use” state, the spring is compressed between two structures of the pump 103 such as the cover 616 and base plate 615. When the cover opening button 610 is pressed, the lever 601 and cover 616 are no longer bound to the body casing 617, and the compressed spring can release stored energy and return to its natural position by pushing the cover 616 away from the base plate 615. In other exemplary embodiments, other actuating mechanisms such as buttons or electrically operated motors may be used in place of lever 601.

In an exemplary embodiment of a constant pressure syringe pump 103, once opened, a user may load a pump-specific syringe 618 filled with medication (not shown) that is unique to the patient's treatment needs. The syringe 618 is connected to an administration set (i.e., a subcutaneous needle set 101 or an intravenous infusion set 201) specific to user treatment needs. The syringe 618 is fitted such that the syringe flange sits securely within the syringe flange receptor 612 such that the extended syringe plunger 620 can be received by the syringe plunger receptor 604 which is connected to the negator carriage 603. The syringe plunger receptor 604 is a protruding extension of negator carriage 603 and does not interfere with any other attached component of the pump 103. The syringe flange receptor 612 is fixed to base plate 615 such that a fully extended syringe plunger 620 of the pump-specific syringe 618 can fit between the syringe flange receptor 612 and the syringe plunger receptor 604. In some exemplary embodiments, the negator carriage 603 may be manually moved back away from the syringe flange receptor 612 such that the syringe 618 may fit within the pump 103.

In an exemplary embodiment, when the pump 103 is not in use, the negator carriage 603 may freely move, within the allowable physical limits, in the direction of the compact (triple) track rail 611. The contact between the negator carriage 603 and the compact (triple) track rail 611 is a low-friction material to enable gliding. Low-friction gliding can be achieved in several manners including the use of ball bearing track contacts (not shown) or other methods.

In the exemplary embodiment, the negator carriage 603 symmetrically houses two specified force negators 602 (also called constant force springs). The negators 602 are fitted onto posts (not shown) of negator carriage 603 using low-friction bearings (not shown) such that negators 602 do not exhibit drag or high frictional forces on the negator carriage 603 when active. The negators 602 are positioned such that they are mirrored about the midline long axis of the negator carriage 603. The negators 602 are placed such that their inner diameters are positioned substantially in parallel to the base plate 615. The negators are further positioned such that when unspooled, the internal surface of both negators 602 will face towards the compact (triple) track rail 611. Further, the negators 602 are symmetrically positioned onto the negator carriage 603 such that when active, the negators 602 do not exhibit unnecessary torsional forces.

In an exemplary embodiment, the negators 602 are secured onto negator carriage 603 with a carriage covering plate. The negators 602 that are attached to the negator carriage 603 are symmetrically positioned onto the compact (triple) track rail 611 such that the negators 602 unspooling direction points in the direction of the compact (triple) track rail 611.

In an exemplary embodiment, between the syringe plunger receptor 604 and the syringe flange receptor 612 is a negator loading carriage 604 that is symmetrically positioned and connected to the compact (triple) track rail 611 similarly to the negator carriage 603. The height of the negator loading carriage 605 is positioned such that it does not interfere with the syringe plunger 620. The negator loading carriage 605 provides two symmetric holes that are specifically placed such that the attachment holes of each negator 602 aligns with the holes of the negator loading carriage 605 such that when connected to the holes of the negator loading carriage 605 and then unspooled, each negator 602 is parallel to the compact (triple) track rail 611. Further, the height of the holes of the negator loading carriage 605 and the height of the negators 602 on the negator carriage 603 is designed such that the negators 602, when unspooled, maintain a substantially parallel configuration to the base plate 615 so as not to introduce unnecessary torsional forces.

In an exemplary embodiment, once the syringe 618 is loaded and secured such that the face of the syringe plunger 620 is securely held within the syringe plunger receptor 604 and the syringe flange is securely held within the syringe flange receptor 612, the cover 616 may be closed such that the cover opening button 610 is reset. The lever 601 is now at a different angle (not illustrated), rotated about the lever attachment point 613 from its starting position when the pump 103 is not in use. The angle of the lever 601 is dependent on the linkage between the lever 601 and the component(s) that move the negator loading carriage 605, such that the negators 602 can be loaded for pump 103 use.

In an exemplary embodiment, the lever 601 is connected to a belt carriage 609 via a linking arm. The linking arm connects to the lever 601 via a lever connection, such that when connected to the belt carriage 609 on the other end the desired lever 601, an activation force and stroke quantity is achieved. The linking arm is connected to the lever 601 and belt carriage 609 such that the linking arm is parallel to both the lever 601 and belt carriage 609. Further, the belt carriage 609, and thus the distal end of the linking arm, is disposed after the negator loading carriage 605 such that, visually, the negator loading carriage 605 sits between the negator carriage 603 and the belt carriage 609.

In an exemplary embodiment, the belt carriage 609 attaches onto the elevated track 661 of the compact (triple) track rail 611 via a track connection, similarly to the negator carriage 603 and the negator loading carriage 605. The belt carriage 609 is placed on an elevated track (not labeled) of the compact (triple) track rail 611 such that it does not interfere in with the movement of negator carriage 603 and the negator loading carriage 605, which ultimately allows the width of pump 103 to be desirably smaller. The belt carriage 609 has one face equally distanced unidirectional teeth distributed across the length of the face. The opposing face of the belt carriage 609, the smooth inside belt surface, is smooth throughout. The unidirectional direction teeth of belt carriage 609 grips onto opposing unidirectional teeth of the belt 607. The belt carriage 609 grips the entire width of the belt 607. The belt carriage 609 and belt 607 have opposing unidirectional teeth, similar to unidirectional ratchet mechanisms, such that the belt 607 can be moved by the belt carriage 609 in one direction, due to the opposing unidirectional teeth, but be fully unengaged when moved in the opposite direction as a result of the unidirectional teeth releasing (i.e., not gripping) one another. In some exemplary embodiments, the belt carriage 609 grips the full width of the belt 607.

In an exemplary embodiment, similar to the belt carriage 609, the negator loading carriage 605 has opposing unidirectional teeth to the belt 607 and grips the belt 607 in a similar fashion as the belt carriage 609. In some exemplary embodiments, only one side of negator loading carriage 605 grips onto the belt 607. As such, the unidirectional teeth of both the belt carriage 609 and the negator loading carriage 605 are in the same direction.

In an exemplary embodiment, the belt 607 is positioned onto four posts placed on the perimeter corners of the compact (triple) track rail 611, see FIG. 6B, where the belt 607 is positioned at the corners of the compact (triple) track rail 611 as an indication of these posts. These posts are each fitted with a belt roller 606. The belt rollers 606 are made of low-friction material and are allowed to freely rotate around the posts on the perimeter corners of the compact (triple) track rail 611. The belt 607 is placed onto the four posts on the perimeter corners of the compact (triple) track rail 611 such that the smooth face of the belt 671 is in direct contact with all four belt rollers 606 and that the unidirectional teeth 673 of the belt 607 are facing away from the compact (triple) track design 611. In some exemplary embodiments, the belt 607 fits onto all four belt rollers 606 such the belt 607 is snug onto the belt rollers 607 such that it does not fall off when the pump 103 is moved, but not too snug such that the belt 607 cannot easily be rotated around the belt rollers 606. As such, the length of the belt 607 is dependent on the perimeter of the four belt rollers 606. Further, the belt 607 is placed such that base plate 615 cannot interfere with the rotation of the belt 607.

In an exemplary embodiment, when the lever 601 is fully pressed down, the linking arm connected to the belt carriage 609 moves the belt carriage 609 forward. As a result, the unidirectional teeth of the belt carriage 609 grip the opposing unidirectional teeth of the belt 607 thus causing the belt to move. As a result, and simultaneously, the unidirectional teeth of the belt 607 grip the opposing unidirectional teeth of the negator loading carriage 605. As a result, the negator loading carriage 605 is pulled towards the direction of the syringe 618 thus causing the negators 602 to unspool. The negator carriage 603 is limited in motion as a result of the opposing force of the syringe plunger 620 as a result of the administration set (i.e., the subcutaneous needle set 101 or intravenous infusion set 201) being closed/blocked or having high flow due to high flow restrictive administration sets and/or high fluid viscosity.

In an exemplary embodiment, the lever 601 was pressed once, so the negators 602 were unspooled to a certain length. However, this is not the negator 602 unspooling length required to dispense the full 60 ml volume of the specified syringe 618. As the lever 601 was pressed once, three more strokes are required to unspool the negators 602 to the length required to dispense 60 ml volume of the specified syringe 618.

In an exemplary embodiment, the user then returns the lever 601 to the fully opened angle position (not shown), which may be aided by the spring (not shown). Moving the lever 601 in this direction moves the linking arm and the attached belt carriage 609 in the same direction. As a result, the belt carriage 609 unidirectional teeth no longer grip the belt 607 allowing the belt carriage 609 to return to the starting position (not labeled). The lever 601 can be pressed three more times to unspool the negators 602 to the length required to dispense the fully 60 ml volume of the specified syringe 618.

In an exemplary embodiment, during dispensing the lever 601 will be down similar to the “not in use” position. The belt 607 grips and maintains the negator loading carriage 605 in a fixed location. As such, the force of the negators 602 attempting to re-spool causes the negator carriage 603 and the syringe plunger receptor 604 to move towards the syringe 618. As a result, the force of the negators 602 acts upon the syringe plunger 620 causing the syringe plunger 620 to dispense the contents of the syringe 618 once the drug path is allowed to flow. In some exemplary embodiments, the components are distanced such that the total allowable volume of the syringe 618 is dispensed.

In an exemplary embodiment, once the contents of the syringe 618 are fully dispensed, the belt release clip 608 may be pressed to push unidirectional teeth of the belt 607 out-of-line with the opposing unidirectional teeth of the negator loading carriage 605 such that negator carriage 603, syringe plunger receptor 604 and negator loading carriage 605 can freely be pushed back towards the starting position such that the syringe 618 can easily be removed and the pump 103 can be used again. The belt release clip 608 may be pressed while dispensing the syringe 618 as deemed necessary by the user, thus stopping the infusion. Doing so releases the belt 607 from the negator loading carriage 605, which may cause the negator loading carriage 605 to travel back towards the negator carriage 603 as a result of the syringe plunger 620 limiting the motion of negator carriage 603 for reasons explained previously. As a result, part damage or louds unpleasant noises may occur. To reduce this, a cushioned brake may be placed between negator carriage 603 and the negator loading carriage 605. The cushioned brake does not interfere with any motion.

In an exemplary embodiment, the belt grips 614 placed on the base plate 615 act as mechanicals supports for pressing the belt release clip 608 and cover opening button 610 and as such are appropriately placed to achieve said support.

In an exemplary embodiment, the lever 601 and cover 616 can be closed post-pump use, thus resetting the cover opening button 610.

Table 1 below shows exemplary required lengths of series tubing 110 at a specified inner diameter required to calibrate the flow dials on the variable flow rate controller 107 (from FIG. 1A). The infusion fluid of Table 1 is specific to 20% immunoglobulins (i.e., Hizentra®) dispensed with a constant pressure source of 13.5 psi and whose viscosities may range from 13-17 centipoises. Given the needle 140 length (0.98″-1.05″) and inner diameter (0.0104″-0.0135″) and needle tubing 110 length (18″-26″) and inner diameter (0.038″-0.042″) remain constant between subcutaneous administration sets 101 only the length of the series tubing 110 must be changed, once an inner diameter is selected, to calibrate the variable flow rate controller 107, such that the maximum flow rate in the provided example is 60 ml/hr per the number of needles 140 within a needle set 101. Once a series tubing 110 inner diameter is selected, the series tubing 110 length required to maintain the flow dials on the variable flow rate controller 107 within calibration can be determined. Simply, the series tubing 110 length is determined such that flow rate (of the variables in the provided example) dispensed from each needle within the needle set 101 is 60 ml/hr when the variable flow rate controller 107 is set to the maximum position. Of course, a skilled artisan will appreciate that specific flow rates can be achieved with numerous other combinations of tubing and needle lengths and diameters besides the examples shown in Table 1 below.

TABLE 1

| Subcutaneous | Series Tube | Series Tube | Maximum |
|---|---|---|---|
| Administration | Length | Inside Diameter | Flow Rate |
| Set Type | (in) | (in) | (mL/hr) |
| 1-Needle | 8.5-12 | 0.015-0.020 | 60 |
| 2-Needle | 11.5-17 | 0.020-0.025 | 120 |
| 3-Needle | 7.5-12 | 0.020-0.025 | 180 |
| 4-Needle | 5.5-8.5 | 0.020-0.025 | 240 |

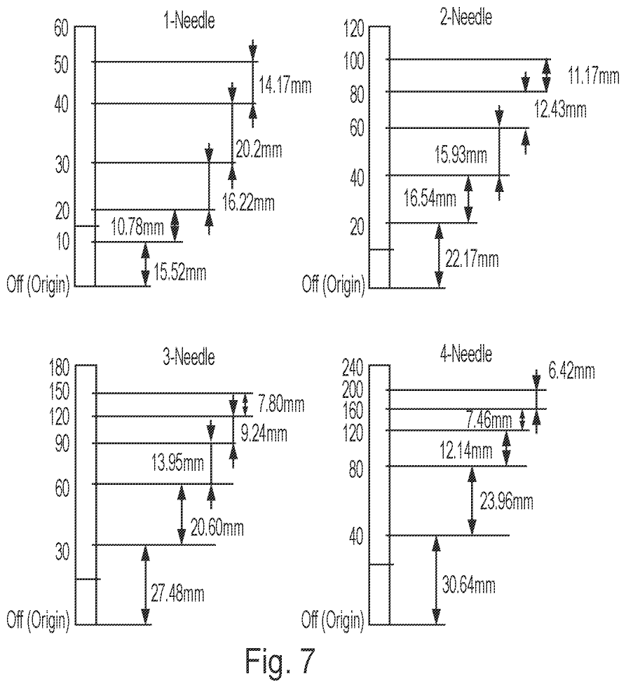

FIG. 7. shows the calibrated flow dials of the variable flow rate controller 107 for 1, 2, 3 and 4-needle administration (needle) set 101 for flow rates of 10, 20, 30, 40, 50 and 60 ml/hr for infusion fluid and infusion and administration set parameters provided in the example presented in Table 1. For the desired flow rate to achieve the lengths and diameters of fluid-pathed components (i.e. the needle, needle tubing, series tubing) must be known. These values may be determined experimentally or optically. Optical methods of determination include direct measurements of inner diameters using optical tools such as compound microscopes. Experimentally, the flow rate can be measured fluidically or using air measurement methods such as flow meter systems. Given the length of the fluid-pathed component and the experimentally measured flow rate the inner diameter of the fluid-pathed component can be calculated using the Hagen-Poiseuille equation (HPE).

The HPE can be used to determine the flow rate of a fluid, with a viscosity, given the length and radius of fluid-pathed components (i.e. the needle tubing) within the administration set, and the differential pressure between the pressure source (i.e. the infusion driver) and the patient's infusion anatomic site. The HPE may be rewritten to solve for any of its variables, including inner diameter of the fluid pathed components. To use the HPE, the following assumptions must be met: the fluid is incompressible, Newtonian, is not accelerating within the administration set, is in laminar flow through the fluid-pathed components of the administration set that maintain a constant circular cross-sectional area and has a length that is substantially larger than its diameter.

Given the above, the HPE can be written as equation (1) below:

$$Q = \frac{Δ p π {R}^{4}}{8 L μ} \qquad ( 1 )$$

where:
Q is the volumetric flow rate of the infusion fluid;
Δp is the differential pressure between the pressure source and the patient's infusion anatomic site;
R is the radius of the fluid-pathed component;
L is the length of the fluid-pathed component; and
μ is the dynamic viscosity of the infusion fluid.

The HPE in combination with a total flow equation (TFE) can be used to determine the flow rate of fluid-pathed flow rate-impacting components within the administration set and the flow rate of the entire administration set.

The flow rates (Q) of each fluid-pathed component must be combined to determine the total flow rate of the administration set. This may be done using the TFE (2) below:

$${Q}_{Total Flow Rate} = \frac{( {Q}_{Series Tubing} ) ( {Q}_{Needle and Needle Tubing} )}{( {Q}_{Series Tubing} + {Q}_{Needle and Needle Tubing} )} \qquad ( 2 )$$

where:
Q_{Total Flow Rate} is the total flow rate of the administration set;
Q_{Series Tubing} is the flow rate of the series tubing 110; and
Q_{Needle and Needle Tubing} is the flow rate of the needle and needle tubing combined.

Knowing the total flow rate of the administration set and of the needle 140 and needle tubing 110 the TPE can be rewritten and solved for the flow rate of the series tubing 110. Given the inner diameter of the series tubing 110 and flow rate the HPE can be used to determine length of the series tubing 110 required to calibrate the administration set such that each needle dispenses a maximum flow rate of 60 ml/hr.

A similar example may be provided for intravenous infusion sets 201 in which the series tubing 210 is at a set inner (0.01780″-0.01820″) whose lengths may be adjusted such that the variable flow rate controller 207 when dispensing an infusion fluid of low viscosity about 1 centipoise (i.e. antibiotics such as Vancomycin®) dispensed with a constant pressure source of 13.5 psi has a maximum flow rate of 300 ml/hr. The flow dials of the variable flow rate controller 207 may be calibrated from 5-300 ml/hr.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

1. An infusion system for delivering an infusion fluid into a patient's anatomic space, the system comprising:
a controller pre-set to deliver a desired flow rate of infusion fluid; and
an administration set matched to the controller, the administration set including a pre-determined number of flow tubes having diameters and lengths selected based upon the desired flow rate and number of infusion sites for a specific infusion fluid treatment.

2. The infusion system of claim 1, wherein the administration set comprises a needle set to subcutaneously deliver the infusion fluid into the patient's anatomic space, and the needle set further comprises:
a pre-determined number of needles having diameters selected based upon the desired flow rate, a number of infusion sites to subcutaneously deliver the infusion fluid into the patient's anatomic space, and the specific infusion fluid to be delivered.

3. The infusion system of claim 2, further comprising:
a substantially constant pressure infusion driver to deliver the infusion fluid; and
wherein the pre-determined number of needles is pre-calibrated to deliver a predetermined flow rate of the specific infusion fluid at a predetermined infusion fluid volume based on the number of needles in the administration set, a flow rate of the flow tubes, and the specific infusion fluid to be delivered.

4. The infusion system of claim 3, wherein the number of needles in the administration set comprises one to eight.

5. The infusion system of claim 3, wherein the controller is configured to be attached to the flow tubes and pre-set to deliver a pre-set flow rate less than or equal to a maximum flow rate for the specific infusion fluid treatment.

6. The infusion system of claim 3, wherein the controller, the flow tubes, and the needles are packaged in a single-use package.

7. The infusion system of claim 1, wherein the administration set comprises an intravenous infusion set to intravenously deliver the infusion fluid into the patient's anatomic space, and the intravenous infusion set further comprises:
a tube to receive the infusion fluid from the infusion driver; and
a connector to receive the infusion fluid from the controller and the tube to deliver the infusion fluid to a catheter at a predetermined flow rate; and
wherein the predetermined flow rate is selected for the specific infusion fluid at a predetermined infusion fluid pressure, and a flow rate of the tube.

8. The infusion system of claim 7, wherein the controller is configured to be attached to the system and pre-set to deliver a pre-set flow rate less than or equal to a maximum flow rate for the specific infusion fluid treatment.

9. The infusion system of claim 1, further comprising:
a tube configured to extend from a source of the infusion fluid to the controller,
wherein the tube has dimensions corresponding to a maximum flow rate for the specific infusion fluid treatment based upon the number of infusion sites.

10.-17. (canceled)

18. A method of manufacturing an infusion system for delivering a specific infusion fluid to a patient's anatomical space, the method comprising the steps of:
matching a flow rate controller to an administration set, wherein the flow controller is pre-set to deliver a desired flow rate of infusion fluid and the administration set includes a predetermined number of flow tubes having lengths and diameters based on the desired flow rate and number of infusion sites for the specific infusion fluid treatment.

19. The method of claim 18, wherein the administration set comprises a needle set to subcutaneously deliver the infusion fluid into the patient's anatomic space, and the method further comprising:
selecting a pre-determined number of needles having diameters selected based on the desired flow rate, a number of infusion sites to subcutaneously deliver the infusion fluid into the patient's anatomic space, and the specific infusion fluid.

20. The method of manufacturing of claim 18, further comprising:
configuring and pre-calibrating a number of needles to deliver the infusion fluid into the patient's anatomic space, and
determining a flow rate of the specific infusion fluid at a pre-determined infusion fluid volume based on the number of needles in the administration set, a flow rate of the flow tubes, and the specific infusion fluid to be delivered.

21. The method of manufacturing of claim 20, further comprising:
configuring the flow rate controller to be attached to the flow tubes;
pre-setting the flow rate controller to deliver a pre-set flow rate less than or equal to a maximum flow rate for the specific infusion fluid treatment.

22. (canceled)

23. (canceled)

24. The method of manufacturing of claim 19, wherein the infusion system is configured to intravenously deliver the infusion fluid into the patient's anatomic space, and the method further comprises:
configuring a tube to receive the infusion fluid from an infusion driver; and
configuring a connector to receive the infusion fluid from the matched flow rate controller and the tube to deliver the infusion fluid to a catheter at a predetermined flow rate selected for the specific infusion fluid treatment at a predetermined infusion fluid pressure based a flow rate of the tube.

25. The method of manufacturing of claim 21, further comprising:
configuring the flow rate controller to be attached to the connector; and
pre-setting the flow rate controller to deliver a pre-set flow rate less than or equal to a maximum flow rate for the specific infusion fluid treatment.

26. The method of manufacturing of claim 18, further comprising:
providing a tube to extend between a source of the infusion fluid to the controller, the tube having dimensions corresponding to a maximum flow rate for the specific infusion fluid treatment based upon the number of infusion sites.

27. An administration set for delivering an infusion fluid into a patient's anatomic space, the administration set comprising:
a pre-determined number of flow tubes having diameters and lengths selected based upon a desired flow rate of a controller and a number of infusion sites for a specific infusion fluid treatment.

28. The administration set of claim 27, further comprising a controller pre-set to deliver a desired flow rate of infusion fluid, and wherein the administration set is matched to the controller.

29. The administration set of claim 27, further comprising a pre-determined number of needles having diameters selected based upon the desired flow rate, a number of infusion sites to subcutaneously deliver the infusion fluid into the patient's anatomic space, and the specific infusion fluid to be delivered.

30. The administration set of claim 28, further comprising:
a tube to receive infusion fluid from a source of infusion fluid; and
a connector to receive infusion fluid from the controller and the tube to deliver the infusion fluid to a catheter at a predetermined flow rate selected for the specific infusion fluid at a predetermined infusion fluid pressure and a flow rate of the tube.
